(19) United States
(12) Patent Application Publication
(10) Pub. No.: US 2019/0354903 A1
(43) Pub. Date: Nov. 21, 2019

(54) RIDE-SHARING ASSISTANCE APPARATUS, RIDE-SHARING ASSISTANCE SYSTEM AND RIDE-SHARING ASSISTANCE METHOD

(71) Applicant: TOYOTA JIDOSHA KABUSHIKI KAISHA, Toyota-shi (JP)

(72) Inventors: Yushi SEKI, Ashigarakami-gun (JP); Ryoko HASHIMOTO, Fuji-shi (JP); Katsuya TERAHATA, Susono-shi (JP); Daiki YOKOYAMA, Gotemba-shi (JP)

(73) Assignee: TOYOTA JIDOSHA KABUSHIKI KAISHA, Toyota-shi (JP)

(21) Appl. No.: 16/368,439

(22) Filed: Mar. 28, 2019

(30) Foreign Application Priority Data
May 16, 2018 (JP) 2018-094818

(51) Int. Cl. G06Q 10/02; G06Q 30/02

(52) U.S. Cl. CPC G06Q 10/02; G06Q 30/0284

(57) ABSTRACT

A ride-sharing assistance apparatus includes a communication circuit capable of communicating with a plurality of terminals and a processor configured to calculate, upon receiving a ride-sharing request signal indicating a request for ride-sharing on a vehicle from at least two terminals of the plurality of terminals via the communication circuit, an evaluation score, for each combination of users of the at least two terminals, that represents compatibility between users included in the combination on the basis of information about a vehicle owned by each of the users of the at least two terminals, determine a combination of users having the highest evaluation score as the combination of users who will share a ride on a vehicle, and send a suggestion signal for suggesting ride-sharing on a vehicle to terminals of the users included in the determined combination of the at least two terminals via the communication circuit.

### FIELD

The present invention relates to a ride-sharing assistance apparatus, a ride-sharing assistance system, and a ride-sharing assistance method for assisting a plurality of users in sharing a ride on a vehicle.

### BACKGROUND

It is preferable for a plurality of users who are going to travel to a same destination to share a ride on a single vehicle to the destination than to separately drive a vehicle to the destination in terms of reducing traffic congestion and exhaust gases. Accordingly, techniques have been proposed for assisting a plurality of users in sharing a ride on a single vehicle (for example, refer to Japanese Unexamined Patent Publication (Kokai) No. 2002-140399 and Japanese Unexamined Patent Publication (Kokai) No. 2005-182146).

For example, an automobile ride-sharing supporting system disclosed in Japanese Unexamined Patent Publication (Kokai) No. 2002-140399 performs a process of matching driving plan information input from a driver side terminal with ride-sharing request information input from a rider side terminal, and sends a meeting place and a meeting time to each terminal. When the system receives that the ride-sharing has been completed from each terminal, the system pays points owned by the rider to the driver.

In a transfer route guidance system disclosed in Japanese Unexamined Patent Publication (Kokai) No. 2005-182146, a route guidance server registers an itinerary of a user input through a first user terminal in advance, and continually accepts registration of car-sharing information from a second user terminal of a car-sharing provider. The route guidance server calculates, on the basis of the itinerary of the user, the car-sharing information, and available transportation means, at least one travel route to a destination, and presents the travel route on the first user terminal. When a travel route including use of car-sharing at least in part is selected from the at least one travel route, the route guidance server generates information prior to agreement with respect to the car-sharing, and sends the information to the first and the second user terminals. When agreement with respect to the car-sharing is obtained from the first and the second user terminals on the basis of the information prior to agreement, the route guidance server authenticates the user and the car-sharing provider to send information after agreement with respect to the car-sharing to the first and the second user terminals.

### SUMMARY

When a plurality of users share a ride on a single vehicle, if compatibility between the users is not good, users who shared the ride may feel stress. In the technique according to Japanese Unexamined Patent Publication (Kokai) No. 2002-140399, an optimum combination of a driver and a person requesting ride-sharing is searched on the basis of closeness between a departure place of the driver and a meeting place, closeness between a departure time of a driver and a desired arrival time of a rider, and matching between desired minimum points of a driver and maximum payable points of a rider; however, compatibility between a driver and a fellow passenger is not known until they actually share a ride. In the technique according to Japanese Unexamined Patent Publication (Kokai) No. 2005-182146, the information prior to agreement, which is sent to each terminal before the user and the car-sharing provider agree to car-sharing, merely includes information about a partner of the car-sharing; compatibility between the user and the car-sharing provider is not known until they actually share a ride.

Therefore, an object of the present invention is to provide a ride-sharing assistance apparatus that can appropriately determine a combination of users who share a ride on a vehicle.

As one aspect of the present invention, a ride-sharing assistance apparatus is provided. The ride-sharing assistance apparatus includes: a communication circuit capable of communicating with a plurality of terminals; and a processor configured to calculate, upon receiving a ride-sharing request signal indicating a request for ride-sharing on a vehicle from at least two terminals of the plurality of terminals via the communication circuit, an evaluation score, for each combination of users of the at least two terminals, that represents compatibility between users included in the combination on the basis of information about a vehicle owned by each of the users of the at least two terminals, determine a combination of users having the highest evaluation score as the combination of users who will share a ride on a vehicle, and send a suggestion signal for suggesting ride-sharing on a vehicle to the terminals of the users included in the determined combination of the at least two terminals via the communication circuit.

In the ride-sharing assistance apparatus, the information about the vehicle includes information indicating classes of vehicles, and the processor preferably sets the evaluation score for a combination of users to be higher as vehicles of the users belong to classes close to each other.

In this case, the processor preferably sets the evaluation score to be lower as a heart rate or a blood pressure of a user who is included in a combination of users for which the evaluation score is calculated and who will share a ride on a vehicle is higher.

Alternatively, the processor preferably calculates the evaluation score by performing a weighted addition of a first score, obtained on the basis of information about a vehicle owned by each of users included in a combination of users for which the evaluation score is calculated, which becomes higher as comfort for each user is better, and a second score, which becomes higher as convenience for each user when the users included in the combination share a ride on a vehicle is greater.

In this case, with respect to the combination of users for which the evaluation score is calculated, the processor is further configured to search for a section for which the users share a ride on the basis of a current location and a destination of each of the users included in the combination; when a length of the searched section or an estimated time required for the searched section is less than a predetermined threshold, the processor preferably sets a higher weight for the second score than that for the first score; on the other hand, when the length of the searched section or the estimated time required for the searched section is greater than the predetermined threshold, the processor preferably sets a higher weight for the first score than that for the second score.

According to another aspect of the present invention, a ride-sharing assistance system including a plurality of terminals and a ride-sharing assistance apparatus capable of communicating with each of the plurality of terminals is provided. In the ride-sharing assistance system, the ride-sharing assistance apparatus calculates, upon receiving a ride-sharing request signal indicating a request for ride-sharing on a vehicle from at least two terminals of the plurality of terminals, an evaluation score, for each combination of users of the at least two terminals, that represents compatibility between users included in the combination on the basis of information about a vehicle owned by each of the users of the at least two terminals, determines a combination of users having the highest evaluation score as the combination of users who will share a ride on a vehicle, and sends a suggestion signal for suggesting ride-sharing on a vehicle to the terminals of the users included in the determined combination of the at least two terminals.

According to still another aspect of the present invention, a ride-sharing assistance method in a ride-sharing assistance system including a plurality of terminals and a ride-sharing assistance apparatus capable of communicating with each of the plurality of terminals is provided. The ride-sharing assistance method includes: sending, by at least two terminals of the plurality of terminals, a ride-sharing request signal indicating a request for ride-sharing on a vehicle to the ride-sharing assistance apparatus; calculating by the ride-sharing assistance apparatus, on the basis of the information about a vehicle owned by each of the users of the at least two terminals from which the ride-sharing request signal is received, an evaluation score, for each combination of users of the at least two terminals, that represents compatibility between users included in the combination; determining, by the ride-sharing assistance apparatus, a combination of users having the highest evaluation score as the combination of users who will share a ride on a vehicle; and sending, by the ride-sharing assistance apparatus, a suggestion signal for suggesting ride-sharing on a vehicle to the terminals of the users included in the determined combination of the at least two terminals.

The ride-sharing assistance apparatus according to the present invention provides an advantageous effect that a combination of users who share a ride on a vehicle is appropriately determined.

### BRIEF DESCRIPTION OF DRAWINGS

FIG. 1 is a schematic configuration diagram of a ride-sharing assistance system according to one example embodiment.

FIG. 2 is a diagram illustrating an example of a situation in which a ride-sharing assistance process is executed.

FIG. 3 is a sequence diagram with respect to the ride-sharing assistance process.

FIG. 4 is a schematic configuration diagram of a server that is an example of a ride-sharing assistance apparatus.

FIG. 5 is an operation flowchart of processing of a portion associated with the server of the ride-sharing assistance process.

FIG. 6 is a diagram illustrating an example of determination of a score for each item included in personal information and information about a vehicle.

FIG. 7 is a schematic configuration diagram of a terminal.

FIG. 8A is a diagram illustrating an example of a display screen displayed on a user interface of a terminal of a user who gets a ride.

FIG. 8B is a diagram illustrating an example of a display screen displayed on a user interface of a terminal of a user who provides a vehicle.

FIG. 9 is diagram illustrating an example of a set of weighting factors for each of a case where emphasis is placed on comfort and a case where emphasis is place on convenience.

### DESCRIPTION OF EMBODIMENTS

With reference to figures, a ride-sharing assistance apparatus, a ride-sharing assistance system including the ride-sharing assistance apparatus, and a ride-sharing assistance process executed by the ride-sharing assistance system will be described below. The ride-sharing assistance system includes a plurality of terminals and a ride-sharing assistance apparatus, and the ride-sharing assistance apparatus evaluates, when the ride-sharing assistance apparatus is informed by at least two terminals that users of the at least two terminals travel to a same destination, compatibility between the users on the basis of personal information of the user of each terminal and information about vehicles owned by the users, and determines a combination of users who will share a ride on a single vehicle on the basis of the evaluation result. The ride-sharing assistance system receives points corresponding to an amount of a price for the ride-sharing from the user who shared the ride, and pays, to the driver, i.e., the user of the vehicle used for the ride-sharing, points after subtracting a service charge from the received points.

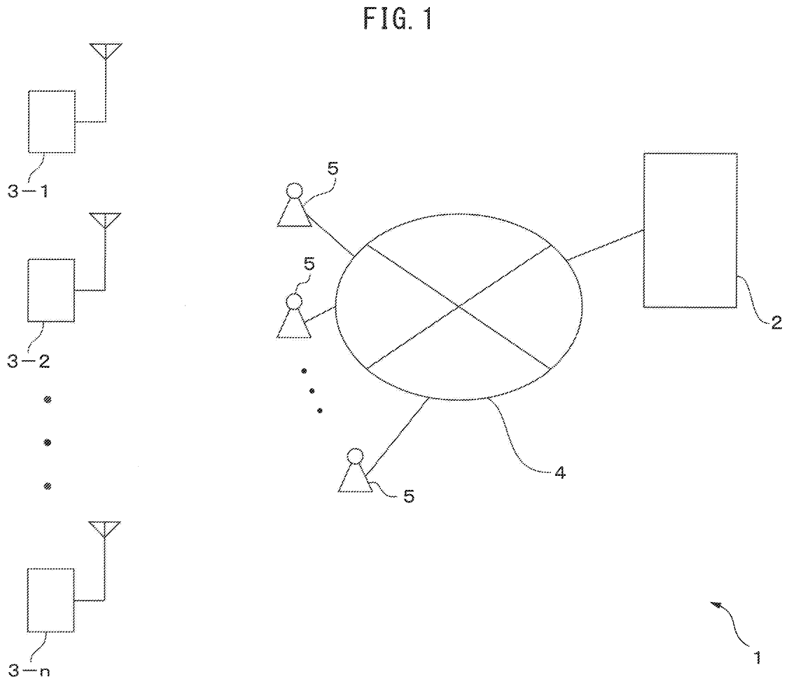

FIG. 1 is a schematic configuration diagram of the ride-sharing assistance system according to one example embodiment. The ride-sharing assistance system 1 includes a server 2, which is an example of the ride-sharing assistance apparatus, and a plurality of terminals 3-1 to 3-n (n is an integer of three or greater). The server 2 and the terminals 3-1 to 3-n are configured to be capable of communicating with each other via a communication network 4, for example, constructed by optical communication lines.

The server 2 is connected to the communication network 4, for example, via a gateway (not illustrated). The server 2 stores personal information of each user of the terminals 3-1 to 3-n, information about vehicles owned by the users, and point information representing a balance of points that indicates an economic value owned by the user. The server 2 executes processing of a portion associated with the server 2 of the ride-sharing assistance process. In other words, when there are ride-sharing requests for traveling to a same destination, which has been informed by at least two terminals of the terminals 3-1 to 3-n (assuming that at least one of the users of the terminals by which the requests have been informed has the a vehicle to be used for the ride-sharing, i.e., a will to be a driver of the vehicle for the ride-sharing), the server 2 calculates an evaluation score representing compatibility between the users with reference to personal information of each user of the terminals by which the requests have been informed and information about vehicles owned by the users, and determines a combination of users who will share a ride on the basis of the evaluation score. The server 2 then suggests ride-sharing and sends a suggestion signal including identification information and personal information of the user who will be a ride-sharing partner, information for identifying a vehicle to be used for the ride-sharing, a meeting place and a meeting time, and the like to the terminal of each user included in the combination. Subsequently, when the server 2 receives an acceptance signal for accepting the suggestion for the ride-sharing from each of the users to whom the ride-sharing has been suggested, the server 2 receives points corresponding to an amount of the price for the ride-sharing from each user who shared the ride, and pays, to the user of the vehicle used for the ride-sharing, points after subtracting a service charge from the received points.

Each of the terminals 3-1 to 3-n is a portable terminal having a wireless communication function, for example, a mobile phone or a tablet computer. The terminals 3-1 to 3-n may be the same type of devices; alternatively, the terminals 3-1 to 3-n may include devices of different types from each other. Each of the terminals 3-1 to 3-n accesses a wireless base station 5 connected to the communication network 4, for example, via a gateway (not illustrated) or the like to connect to the communication network 4 via the wireless base station 5. Each of the terminals 3-1 to 3-n can send a current location and a destination of a user generated in response to an operation by the user as well as a ride-sharing request signal for informing that there is a ride-sharing request or an acceptance signal, or the like to the server 2 via the communication network 4, or receive a suggestion signal or the like from the server 2 via the communication network 4. In the following description, for the sake of convenience, a terminal that sends the ride-sharing request signal may be referred to as a target terminal.

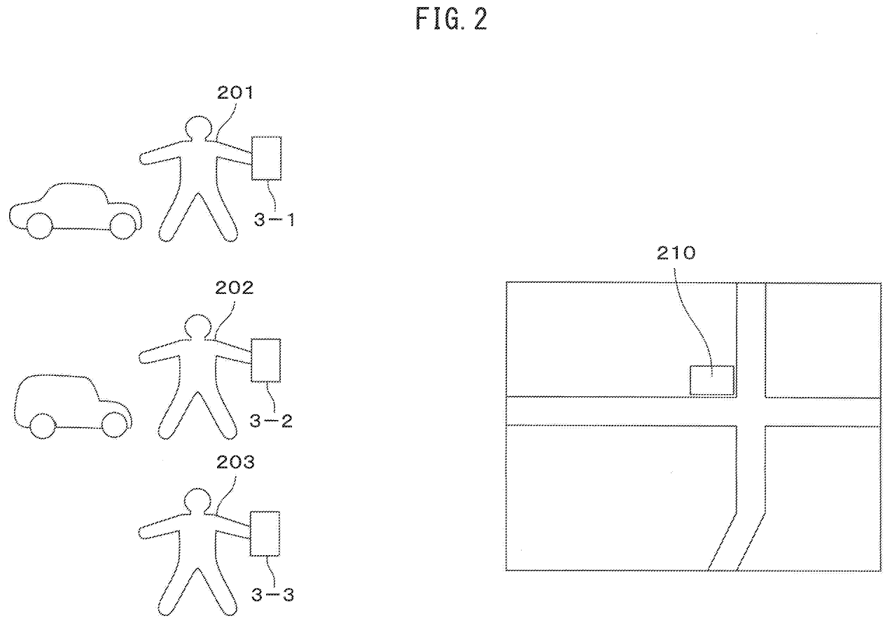

FIG. 2 is a diagram illustrating an example of a situation in which the ride-sharing assistance process is executed. In this example, each of a user 201 of the terminal 3-1, a user 202 of the terminal 3-2, and a user 203 of the terminal 3-3 is going to travel to a same destination 210. The user 201 and the user 202 have an intention to provide vehicles owned by them for ride-sharing. Therefore, the server 2 calculates, for a combination of the user 201 and the user 203 and a combination of the user 202 and the user 203, evaluation scores representing compatibility, and suggests ride-sharing for the combination having the higher evaluation score.

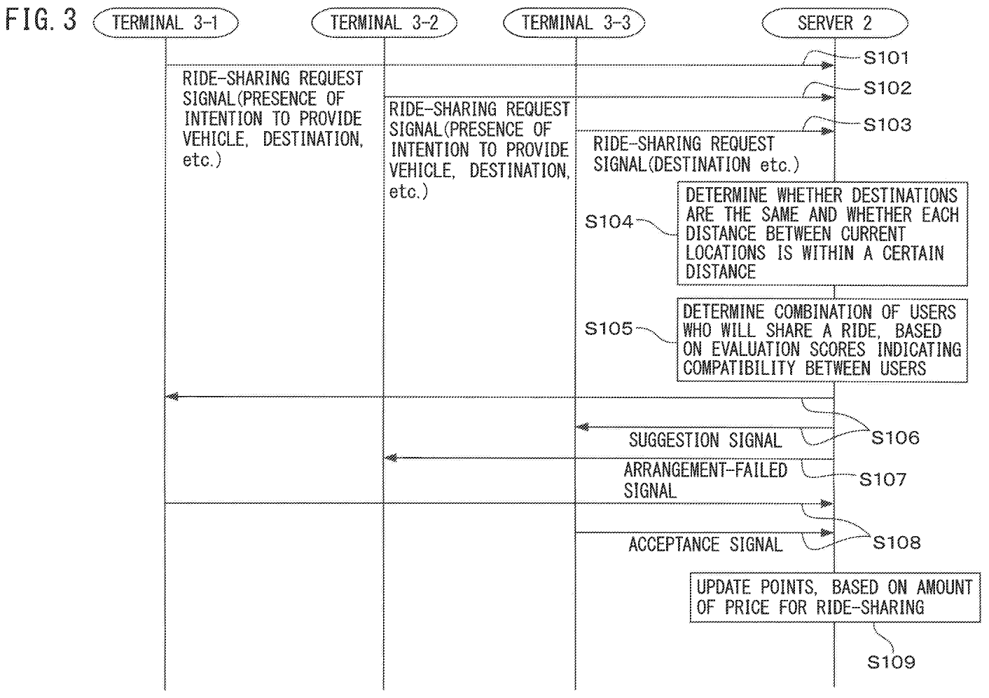

FIG. 3 is a sequence diagram with respect to the ride-sharing assistance process. In the sequence diagram illustrated in FIG. 3, communication between each terminal and the server is performed, as described above, via the communication network 4.

The user of the terminal 3-1 operates the terminal 3-1 to start an application associated with the ride-sharing assistance process, and performs, through the application, an operation for informing that the user may share a ride and that the user may provide a vehicle owned by the user for the ride-sharing. The terminal 3-1 then generates a ride-sharing request signal and sends the ride-sharing request signal to the server 2 (step S101). The user of the terminal 3-2 also operates the terminal 3-2 to start an application associated with the ride-sharing assistance process, and performs, through the application, an operation for informing that the user may share a ride and that the user may provide a vehicle owned by the user for the ride-sharing. The terminal 3-2 then generates a ride-sharing request signal and sends the ride-sharing request signal to the server 2 (step S102). Furthermore, the user of the terminal 3-3 also operates the terminal 3-3 to start an application associated with the ride-sharing assistance process, and performs, through the application, an operation for informing that the user requests ride-sharing. The terminal 3-3 then generates a ride-sharing request signal and sends the ride-sharing request signal to the server 2 (step S103). The ride-sharing request signal includes a current location and a destination of a user. The destination and the current location are represented by, for example, a facility name, an address, or a combination of longitude and latitude.

When timings at which the ride-sharing request signal is received from the terminals 3-1 to 3-3 are within a certain period of time (for example, three minutes to ten minutes), the server 2 determines whether the destinations included in the respective ride-sharing request signals received from the terminals are the same, and determines whether each distance between current locations of the users of the terminals is equal to or less than a certain distance (for example, 1 km to 2 km) (step S104). For example, the server 2 determines, when the facility names or the addresses of the destinations included in the respective ride-sharing request signals received from the terminals are the same, that the destinations are the same. Alternatively, the server 2 may calculate a distance between the destinations included in the respective ride-sharing request signals received from the terminals and determine that, when the distance is equal to or less than a predetermined distance threshold (for example, 50 m to 200 m), the destinations are the same.

When the destinations included in the respective ride-sharing request signals received from the terminals are the same and each distance between current locations of the users of the terminals is equal to or less than the certain distance, the server 2 calculates, with reference to personal information of each user of the terminals and information about vehicles owned by the users, evaluation scores representing compatibility between the users, and determines, on the basis of the evaluation scores, a combination of users who will share a ride (step S105). For example, in this case, it is assumed that the evaluation score for the combination of the user 201 and the user 203 is higher than that for the combination of the user 202 and the user 203. Consequently, the server 2 sends a suggestion signal for suggesting ride-sharing to the terminal 3-1 and the terminal 3-3 (step S106). When performing this operation, the server 2 searches for a route from the current location of the user 201 to the destination in such a way that the route includes the meeting place for the user 203, and on the basis of the search result, determines a meeting place and a meeting time. The server 2 then includes identification information and personal information of the user who will be a ride-sharing partner, information for identifying the vehicle to be used for the ride-sharing, the meeting place and the meeting time, and the like in the suggestion signal. On the other hand, to the terminal 3-2 of the user 202 to whom the server 2 does not suggest ride-sharing, the server 2 sends an arrangement-failed signal indicating that arrangement of the ride-sharing has failed (step S107).

When both of the user 201 and the user 203 accept the ride-sharing suggestion, the terminal 3-1 and the terminal 3-3 send an acceptance signal for accepting the ride-sharing to the server 2 (step S108). When the server 2 receives the acceptance signal from each of the terminals to which the suggestion signal has been sent, the server 2 receives points corresponding to an amount of the price for the ride-sharing from the user who shared the ride (in this example, the user 203), and pays, to the user of the vehicle used for the ride-sharing (in this example, the user 201), points after subtracting a service charge from the received points (step S109). Then, the ride-sharing assistance system ends the ride-sharing assistance process.

When the ride-sharing is completed, the terminal of the user who shared the ride may send, in response to an operation by the user, a ride-sharing completion signal for informing that the ride-sharing is completed to the server 2. The server 2 may execute processing for the step S109 after receiving the ride-sharing completion signal. The terminal of the user who shared the ride may include, in the ride-sharing completion signal, a heart rate or a blood pressure of the user measured while the user was sharing the ride on the vehicle.

Details of the server 2 will be described below.

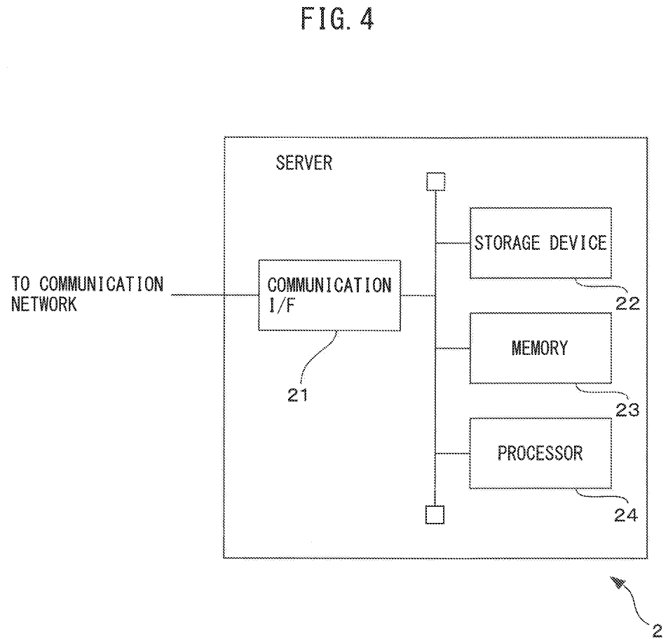

FIG. 4 is a schematic configuration diagram of the server 2. The server 2 includes a communication interface 21, a storage device 22, a memory 23, and a processor 24. The communication interface 21, the storage device 22, and the memory 23 are connected to the processor 24 via a signal line. The server 2 may further include an input device such as a keyboard or a mouse and an output device such as a display.

The communication interface 21 is an example of the communication unit, and includes an interface circuit for connecting the server 2 to the communication network 4. The communication interface 21 is configured to be capable of communicating with each of the terminals 3-1 to 3-n. In other words, the communication interface 21 passes the ride-sharing request signal, the acceptance signal, and the like received from any one of the terminals 3-1 to 3-n via the communication network 4 to the processor 24. The communication interface 21 outputs a suggestion signal and the like received from the processor 24 to the communication network 4.

The storage device 22 is an example of a storage unit, and includes, for example, a hard disk device or an optical recording medium and an access device therefor. The storage device 22 stores, for each of the terminals 3-1 to 3-n, identification information about the terminal, a name and identification information (for example, a membership number of a member eligible for receiving a service in the ride-sharing assistance process) of the user of the terminal, personal information of the user of the terminal, information for identifying a vehicle owned by the user of the terminal (for example, a vehicle license-plate number), and information about the vehicle, in association with each other. The storage device 22 also stores, for each of the terminals 3-1 to 3-n, point information of the user of the terminal. The storage device 22 further stores various types of information used in the ride-sharing assistance process such as map information. The identification information about each terminal may be, for example, a MAC address. In addition, the storage device 22 may also store a computer program for executing the ride-sharing assistance process.

The personal information includes at least one of, for example, age, gender, interests, business, presence or absence of a driving violation record, a heart rate or a blood pressure during past ride-sharing, and a driving pattern of the user. The driving pattern indicates characteristics of driving behaviors of a driver, and is classified into, for example, a sedate type or a sporty type. The driving pattern is determined on the basis of, for example, a number of times that an absolute value of acceleration of a vehicle, which is measured by an acceleration sensor installed in the vehicle, exceeds a predetermined acceleration threshold in a certain period of time or a predetermined traveling distance, a number of times that a rotation amount of a steering, which is measured by a steering angle sensor, exceeds a predetermined rotation amount threshold in a certain period of time or a predetermined traveling distance, or the like. In other words, an error between a value of the number of times set for each driving pattern as described above and a value of the number of times measured for the user as described above is calculated, and a driving pattern for which the error is the smallest is identified as the user's driving pattern. The information about the vehicle includes, for example, a class of the vehicle owned by the user. In addition, the information about the vehicle may include a class of a vehicle owned by the user in the past or a repair record of the vehicle currently owned by the user. Classes of vehicles are defined, for example, according to a size of an engine installed in a vehicle. In this case, the larger the engine size is, more upper the class is. Alternatively, classes of vehicles may be defined according to a selling price of a new vehicle. In this case, the higher the selling price is, more upper the class is. Alternatively, classes of vehicles may be defined according to what is termed as a segment. In this case, more upper the corresponding segment is, more upper the class is.

The memory 23 is another example of the storage unit, and includes, for example, non-volatile semiconductor memory and volatile semiconductor memory. The memory 23 stores various types of data generated during execution of the ride-sharing assistance process.

The processor 24 is an example of the control unit, and includes one or more CPUs (Central Processing Unit) and a peripheral circuit thereof. The processor 24 may further include another arithmetic circuit such as an arithmetic logic unit or a numeric data processing unit. The processor 24 executes processing of the portion associated with the server 2 of the ride-sharing assistance process.

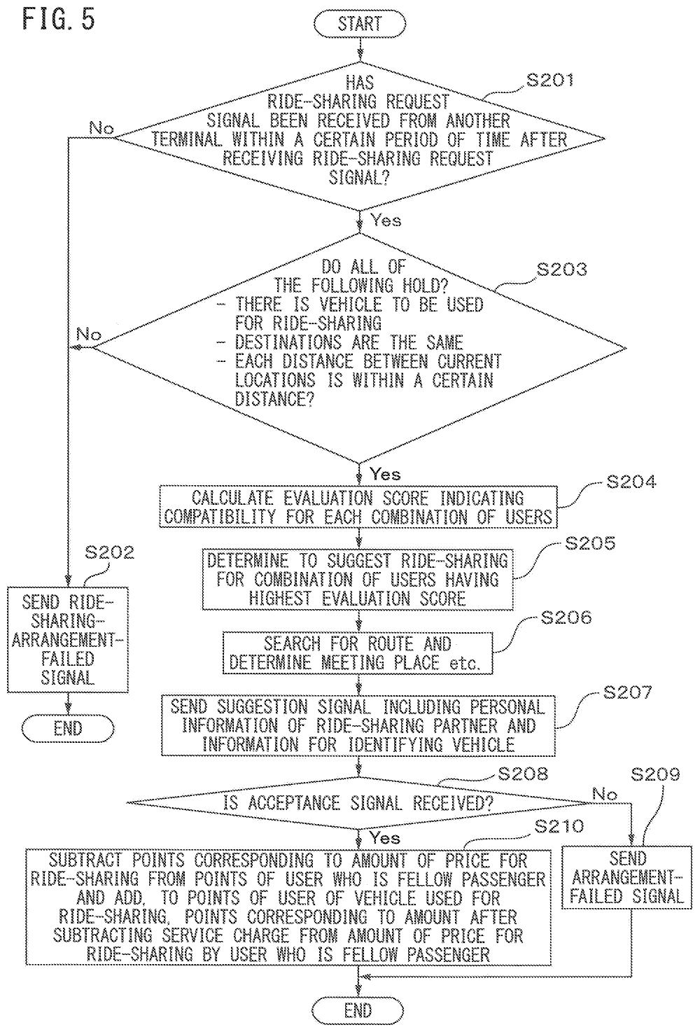

FIG. 5 is an operation flowchart of processing of the portion associated with the server 2 of the ride-sharing assistance process. When the server 2 receives the ride-sharing request signal from any one of the terminals 3-1 to 3-n via the communication network 4 and the communication interface 21, the processor 24 executes processing of the portion associated with the server 2 of the ride-sharing assistance process in accordance with the operation flowchart illustrated in FIG. 5.

After receiving the ride-sharing request signal from any one of the terminals, the processor 24 determines whether the processor 24 has received a ride-sharing request signal from another terminal within a certain period of time (step S201). When the processor 24 has not received a ride-sharing request signal from another terminal within the certain period of time (step S201-No), the processor 24 returns an arrangement-failed signal indicating that arrangement of the ride-sharing has failed to the terminal (target terminal) from which the ride-sharing request signal has been sent via the communication interface 21 and the communication network 4 (step S202). Then, the processor 24 ends the ride-sharing assistance process.

On the other hand, when the processor 24 has received a ride-sharing request signal from another terminal within a certain period of time (step S201—Yes), the processor 24 retrieves, from each of the received ride-sharing request signals, information indicating presence or absence of an intention to provide a vehicle to be used for the ride-sharing, a destination, and a current location, and determines whether there is a vehicle to be used for the ride-sharing, whether the destinations are the same, and whether each distance between the current locations is equal to or less than a certain distance (step S203). When no user has an intention to provide a vehicle to be used for the ride-sharing, when the destinations are different, or when the distance between the current locations is greater than the certain distance (step S203-No), the processor 24 executes processing for the step S202 for each target terminal. In other words, the server 2 sends an arrangement-failed signal to each target terminal. Then, the processor 24 ends the ride-sharing assistance process.

On the other hand, when any one of the users has an intention to provide a vehicle to be used for the ride-sharing, the destinations are the same, and the distance between the current locations is equal to or less than the certain distance (step S203—Yes), the processor 24 calculates, for each combination of the user having an intention to provide a vehicle to be used for the ride-sharing (i.e., a driver) and another user (i.e., a fellow passenger), an evaluation score representing compatibility between the users with reference to personal information of the users and information about vehicles owned by the users (step S204).

When a plurality of users show an intention to provide a vehicle to be used for the ride-sharing, the processor 24 may execute, for each user showing the intention to provide a vehicle to be used for the ride-sharing, processing for the step S204. Alternatively, the processor 24 may execute, with defining one of the users showing an intention to provide a vehicle to be used for the ride-sharing as the fellow passenger and the other as the driver, processing for the step S204.

For example, the processor 24 obtains, for a combination of users for which the evaluation score is calculated, a score for each item included in the personal information and the information about the vehicles, and calculates the evaluation score by performing a weighted addition of scores for the items using a weight factor for each item. In this case, the score for each item is determined in such a way that the score is higher as a difference between users with respect to the item is smaller.

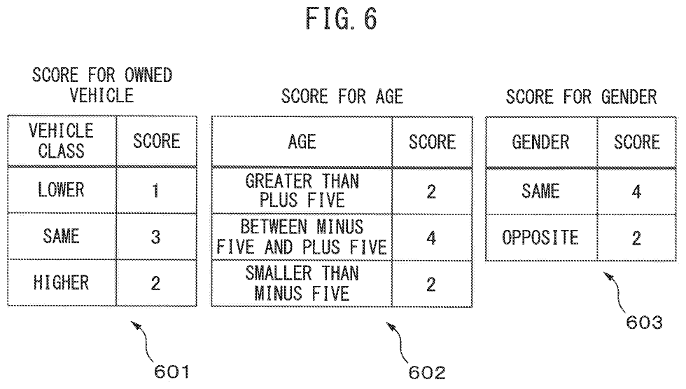

FIG. 6 is a diagram illustrating an example of determination of the score for each item. Tables 601 to 603 respectively show definitions of scores for items related to a class of a vehicle owned by a user, age, and gender. For example, as illustrated in the table 601, with respect to the item related to a class of a vehicle, the score in a case where the class of the vehicle to be used for the ride-sharing is the same as that of a vehicle owned by the user who will be the fellow passenger is the highest (three points), the score in a case where the class of the vehicle to be used for the ride-sharing is higher than that of the vehicle owned by the user who will be the fellow passenger is the second highest (two points), and the score in a case where the class of the vehicle to be used for the ride-sharing is lower than that of the vehicle owned by the user who will be the fellow passenger is the lowest (one point). When the information about the vehicles includes a class of a vehicle owned in the past by the user who will be the fellow passenger, the score for the item related to the class of the vehicle may be obtained on the basis of a class closest to the class of the vehicle to be used for the ride-sharing among the classes of vehicles that the user who will be the fellow passenger currently owns or owned in the past.

As illustrated in the table 602, the item related to age is defined in such a way that the score in a case where a difference between ages of the users is five or less (four points) is higher than that in a case where the difference between ages of the users is greater than five (two points). As illustrated in the table 603, the item related to gender is defined in such a way that the score in a case where genders of the users are the same (four points) is higher than the score in a case where the genders of the users are different (two points).

When the information about the vehicle to be used for the ride-sharing includes presence or absence of a repair record, the score in a case where there is a repair record may be defined to be lower than the score in a case where there is no repair record. With this operation, the processor 24 may reduce a possibility that a vehicle with a repair record is used for the ride-sharing. When the personal information of the user who will be the fellow passenger includes a heart rate during past ride-sharing, the score in a case where the heart rate is equal to or greater than a predetermined threshold may be defined to be lower than that in a case where the heart rate is lower than the predetermined threshold. Generally speaking, when a human feels stress, the heart rate becomes high. Therefore, a user whose heart rate was high during ride-sharing is highly likely to have felt stress during the ride-sharing. By defining the score for the heart rate as described above, the evaluation score for a combination including a user who has felt stress during ride-sharing becomes low, and the possibility of suggesting ride-sharing for the combination will be reduced.

When the personal information includes an item related to presence or absence of a driving violation record, the processor 24 may set, with respect to a user having an intention to provide a vehicle to be used for the ride-sharing, the score for the item in such a way that the score in a case where there is a violation record is lower than that in a case where there is no violation record. This operation increases the possibility of suggesting ride-sharing for the combination including a user who is highly likely to try to drive safely.

The processor 24 determines to suggest ride-sharing for a combination having the highest evaluation score among evaluation scores for combinations of a user having an intention to provide a vehicle to be used for the ride-sharing and another user (S205). The processor 24 then searches for a route from the current location of the user who has shown the intention to provide a vehicle to be used for the ride-sharing to the destination, and determines a meeting place and a meeting time for the user (step S206). In this case, the processor 24 may search for a route, on the basis of the current location of the user who is included in the determined combination and who will be the fellow passenger, in such a way that the route passes a location within a certain range (for example, 100 to 200 m) from the current location of the user and that the time required is the shortest, in accordance with a route search technique such as the Dijkstra's algorithm. The processor 24 also obtains, on the basis of the searched route, the meeting place and the meeting time for the user who will be the fellow passenger. The processor 24 may further search for a route in a case where ride-sharing is not conducted, from the current location of the user who will be the driver to the destination, and estimate the time required based on the route to obtain a difference from the time required based on a route in a case where ride-sharing is conducted. The processor 24 then generates a suggestion signal for informing identification information for identifying the ride-sharing partner (for example, a membership number), personal information of the partner, information for identifying the vehicle to be used for the ride-sharing (for example, a vehicle license-plate number), the meeting place and the meeting time, and the like, and sends the generated suggestion signal to each target terminal via the communication interface 21 and the communication network 4 (step S207). The processor 24 may include only a part of personal information stored in the storage device 22 in the suggestion signal as the personal information of the ride-sharing partner. For example, when the personal information stored in the storage device 22 includes items related to age, gender, interests, and a violation record, the processor 24 may include, in the suggestion signal, only items related to age, gender, and interests. The items of the personal information included in the suggestion signal may be set by the user in advance. This operation prevents disclosure of information that the user does not wish to be disclosed to other users without receiving authorization from the user. When there is a user for whom ride-sharing arrangement has failed, the processor 24 may generate a requested-arrangement-failed signal, and send the requested-arrangement-failed signal to the terminal of the user for whom ride-sharing arrangement has failed via the communication interface 21 and the communication network 4.

The processor 24 determines whether the processor 24 has received an acceptance signal from each of the target terminals within a certain period of time after sending the suggestion signal (step S208). When the processor 24 has not received the acceptance signal from each of the target terminals within the certain period of time (step S208-No), the processor 24 generates an arrangement-failed signal, and sends the generated arrangement-failed signal to each of the target terminals via the communication interface 21 and the communication network 4 (step S209). Then, the processor 24 ends the ride-sharing assistance process.

On the other hand, when the processor 24 has received the acceptance signal from each of the target terminals within the certain period of time (step S208—Yes), the processor 24 reads out point information of a user corresponding to each of the terminals from which the acceptance signal has been sent, i.e., each user who shared the ride, from the storage device 22. The processor 24 then subtracts, from a balance of points of the user who is the fellow passenger, points corresponding to the amount of the price for the ride-sharing, updates the points of the user, and stores the updated points in the storage device 22. Furthermore, the processor 24 adds, to a balance of points of the user of the vehicle used for the ride-sharing, points corresponding to the amount after subtracting the service charge from the amount of the price for the ride-sharing by the user who is the fellow passenger, updates the points of the user of the vehicle, and stores the updated points in the storage device 22 (step S210). Thus, the ride-sharing assistance system ends the ride-sharing assistance process.

According to a variation, in the step S203, instead of determining whether the destinations of the users are the same and each distance between the current locations of the users is equal to or less than the certain distance, the processor 24 may determine whether each of a distance between the route, which has been searched by means of a predetermined search technique, from the current location of the user who will be the driver to the destination and the current location of the user who will be the fellow passenger and a distance between the route and the destination of the user who will be the fellow passenger is equal to or less than a certain distance. When each of the distance between the route from the current location of the driver to the destination and the current location of the user who will be the fellow passenger and the distance between the route and the destination of the user who will be the fellow passenger is equal to or less than the certain distance, the processor 24 may then execute processing for the step S204 and later.

Next, details of the terminals 3-1 to 3-n will be described. With respect to a configuration associated with the ride-sharing assistance process, each terminal may have the same configuration; therefore, only the configuration of the terminal 3-1 will be described below.

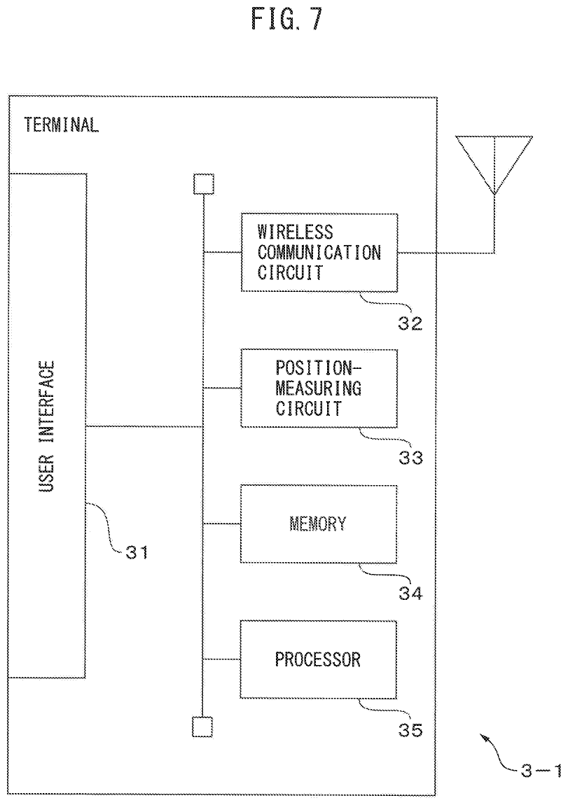

FIG. 7 is a schematic configuration diagram of the terminal 3-1. The terminal 3-1 includes a user interface 31, a wireless communication circuit 32, a position-measuring circuit 33, a memory 34, and a processor 35. The terminal 3-1 may further include a near field wireless communication circuit (not illustrated) for performing wireless communication with a biological sensor, which is attached to a user, for measuring biological information of the user such as a heart rate or a blood pressure of the user.

The user interface 31 includes, for example, a touchscreen display. The user interface 31 generates a signal according to an operation by a user associated with the ride-sharing assistance process (for example, starting of an application associated with the ride-sharing assistance process, input of a destination and presence or absence of an intention to provide a vehicle when requesting ride-sharing, or whether to accept ride-sharing when receiving a suggestion signal), and outputs the signal to the processor 35. In addition, the user interface 31 displays various types of information for display received from the processor 35 (for example, information for identifying a vehicle to be used for the ride-sharing such as a vehicle license-plate number of the vehicle when receiving a suggestion signal, personal information of a ride-sharing partner, a meeting place, or a meeting time).

The wireless communication circuit 32 includes, for example, an antenna and a signal processing circuit for executing various types of processes associated with wireless communication such as modulation and demodulation of a radio signal. The wireless communication circuit 32 receives a downlink radio signal from the wireless base station 5 connected to the communication network 4 via a gateway or the like, and sends an uplink radio signal to the wireless base station 5. In other words, the wireless communication circuit 32 retrieves, from the downlink radio signal received from the wireless base station 5, a signal transmitted from the server 2 to the terminal 3-1 (for example, a suggestion signal), and passes the signal to the processor 35. In addition, the wireless communication circuit 32 generates an uplink radio signal including a signal received from the processor 35 and sent to the server 2 (for example, a ride-sharing request signal or an acceptance signal), and sends the radio signal.

The position-measuring circuit 33 obtains information indicating a location of the terminal 3-1. The position-measuring circuit 33 includes, for example, a receiver for receiving a GPS signal and an arithmetic circuit for calculating the location of the terminal 3-1 from the GPS signal. The position-measuring circuit 33 measures the location of the terminal 3-1 on the basis of the GPS signal. Every time the position-measuring circuit 33 measures the location of the terminal 3-1, the position-measuring circuit 33 informs the processor 35 of the location.

The memory 34 includes, for example, non-volatile readable/writable semiconductor memory and volatile readable/writable semiconductor memory. The memory 34 stores various types of application programs to be executed by the processor 35 and various types of data. The memory 34 stores various types of data associated with execution of the ride-sharing assistance process.

The processor 35 includes one or more CPUs and a peripheral circuit thereof. The processor 35 may further include another arithmetic circuit such as an arithmetic logic unit or a numeric data processing unit. The processor 35 executes processing of a portion associated with the terminal 3-1 of the ride-sharing assistance process. For example, the processor 35 generates, in response to an operation by the user, a signal to be sent to the server 2 (a ride-sharing request signal, an acceptance signal, or the like), and outputs the generated signal to the wireless communication circuit 32.

For example, in a state in which an application associated with the ride-sharing assistance process has been started, when the user requests ride-sharing through the user interface 31 and an operation for inputting a destination and the like is performed, the processor 35 generates a ride-sharing request signal including the input destination and presence or absence of an intention to provide a vehicle to be used for the ride-sharing, a current location of the terminal 3-1 measured by the position-measuring circuit 33, and identification information about the terminal 3-1.

Upon receiving a suggestion signal, the processor 35 retrieves information for identifying the vehicle to be used for the ride-sharing, identification information and personal information of the ride-sharing partner, and the meeting place and the meeting time, which are included in the suggestion signal, and causes the user interface 31 to display these pieces of information. In this case, the processor 35 may display the meeting place together with a map. Upon receiving the suggestion signal, when the user performs an operation indicating that the user accepts the ride-sharing via through user interface 31, the processor 35 further generates an acceptance signal.

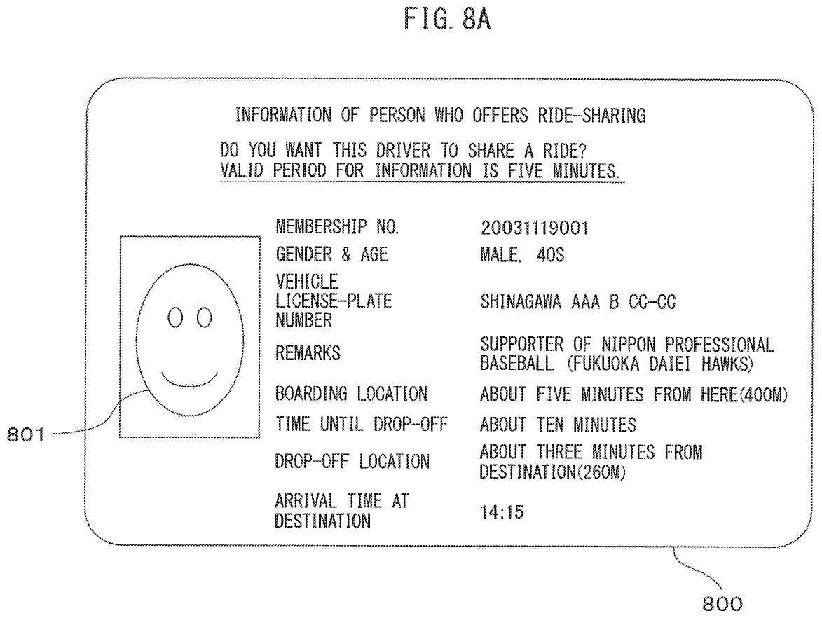

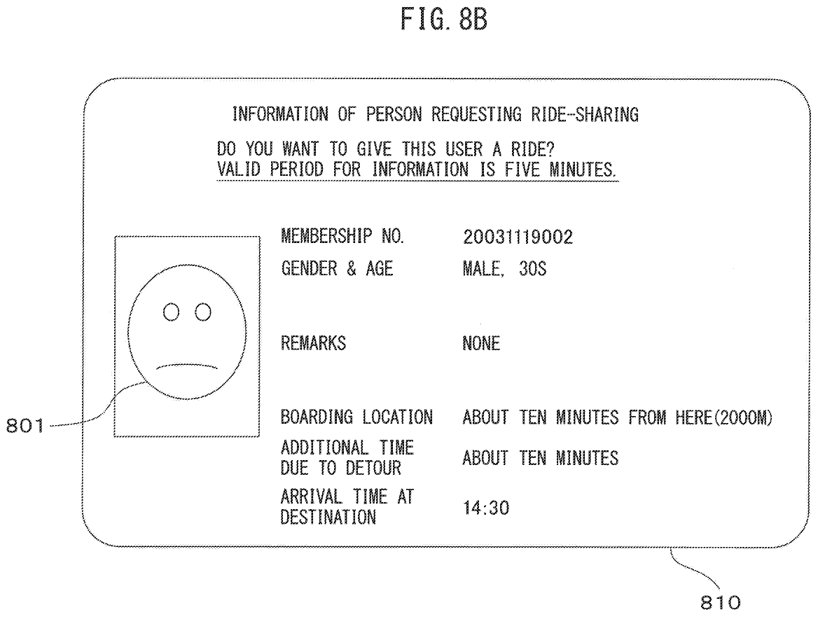

FIG. 8A is a diagram illustrating an example of a display screen, which displays personal information or the like included in the suggestion signal, displayed on the user interface 31 of the terminal of the user who gets a ride, i.e., the user who will be the fellow passenger. In contrast, FIG. 8B is a diagram illustrating an example of a display screen, which displays personal information or the like included in the suggestion signal, displayed on the user interface 31 of the terminal of the user who provides a vehicle, i.e., the user who will be the driver. As illustrated in FIG. 8A, the display screen 800 for the user who will be the fellow passenger displays a valid period for the information, which indicates a time limit for a response to the acceptance signal, the membership number of the user (driver), which is identification information about the user who will be the partner, the age, the gender, the vehicle license-plate number of the vehicle to be used for the ride-sharing, the meeting place, time until the meeting time, and the like. As illustrated in FIG. 8B, the display screen 810 for the user who will be the driver displays the valid period for the information, the membership number of the user (fellow passenger) who will be the partner, the age, the gender, the meeting place, an additional time due to sharing a ride, and the like.

The suggestion signal may further include a face image of the ride-sharing partner. The processor 35 of each terminal that has received the suggestion signal may cause the user interface 31 to display a face image 801 of the ride-sharing partner. With this operation, each user may easily identify the ride-sharing partner when meeting the ride-sharing partner. The face image is stored in advance associated with the identification information about the user, for example, in the storage device 22 of the server 2. Alternatively, each terminal may include a camera (not illustrated), and the processor 35 of each terminal may include a face image acquired by the camera in the ride-sharing request signal.

As described above, the ride-sharing assistance apparatus calculates, on the basis of personal information of users who request ride-sharing and information about vehicles owned by the users, an evaluation score representing compatibility between the users, and determines, on the basis of the evaluation score, a combination of the users who will share a ride on a single vehicle. In particular, the ride-sharing assistance apparatus utilizes, in order to calculate the evaluation score, information about a vehicle owned by each of the users. Therefore, the evaluation score reflects tastes of the users with respect to vehicles. Thus, the evaluation score may appropriately represent the compatibility when the users share a ride on a single vehicle. Consequently, the ride-sharing assistance apparatus can appropriately determine a combination of users who will share a ride on a single vehicle in such a way that each user who share the ride may not feel stress.

According to a variation, when the server 2 calculates the evaluation score for a combination of users, the server 2 may not refer to personal information. Alternatively, when the server 2 calculates the evaluation score for a combination of users, the server 2 may refer to an item related to convenience together with the information about the vehicle owned by each of the users and personal information. For example, the processor 24 of the server 2 may search, for each combination of users for which the evaluation score is calculated, for a route from a current location of a user who provides a vehicle to be used for ride-sharing to a destination for each of a case where the ride-sharing is conducted and a case where the ride-sharing is not conducted, and obtain a difference between an estimated arrival time at the destination from the current location, which is obtained on the basis of the searched route when the ride-sharing is conducted, and an estimated arrival time at the destination from the current location, which is obtained on the basis of the searched route when the ride-sharing is not conducted. As the difference is greater, the processor 24 may set a score for an item related to convenience to be lower. Alternatively, the processor 24 may obtain a waiting time from a time at which the ride-sharing request signal is received from the terminal of the user who will be the fellow passenger to the meeting time at which the vehicle to be used for the ride-sharing reaches the meeting place, and set the score for the item related to convenience to be lower.

According to another variation, a weighting factor for the score for each item when the evaluation score is calculated may be varied according to a length of a section for which users share a ride or an estimated time required for the section for which users share a ride. For example, when the length of the section for which users share a ride or the estimated time required for the section for which users share a ride is short, it is preferable to have higher convenience, such as reaching the destination earlier, even if the users who share the ride feel a little stress. On the other hand, when the length of the section for which users share a ride or the estimated time required for the section for which users share a ride is long, it is preferable that the users who share the ride may not feel stress. Therefore, according to the variation, a set of weighting factors for scores for items in a case where emphasis is placed on convenience and a set of weighting factors for scores for items in a case where emphasis is placed on comfort are stored in the storage device 22 in advance. In the step S204 of the operation flowchart illustrated in FIG. 5, the processor 24 of the server 2 estimates, on the basis of a result of search for a route to the destination, a length of the section for which users will share a ride. When the estimated length of the section for which the users share the ride is equal to or less than a predetermined distance threshold, the processor 24 reads out the set of weighting factors for scores for items for the case where emphasis is placed on convenience from the storage device 22, and calculates the evaluation score using the read set of weighting factors. On the other hand, when the estimated length of the section is greater than the predetermined distance threshold, the processor 24 reads out the set of weighting factors for scores for items for the case where emphasis is placed on comfort from the storage device 22, and calculates the evaluation score using the read set of weighting factors. The distance threshold is set to, for example, 60 km when half or more of the section for which the users share the ride is a section passing through a freeway, otherwise 40 km.

Alternatively, the processor 24 may estimate, on the basis of the result of the search for a route to the destination, a time required for the section for which the users share the ride. In this case, when the estimated time required is equal to or less than a predetermined time threshold, the processor 24 reads out the set of weighting factors for scores for items for the case where emphasis is placed on convenience from the storage device 22, and calculates the evaluation score using the read set of weighting factors. On the other hand, when the estimated time required is greater than a predetermined time threshold, the processor 24 reads out the set of weighting factors for scores for items for the case where emphasis is placed on comfort from the storage device 22, and calculates the evaluation score using the read set of weighting factors. The time threshold is set to, for example, one hour.

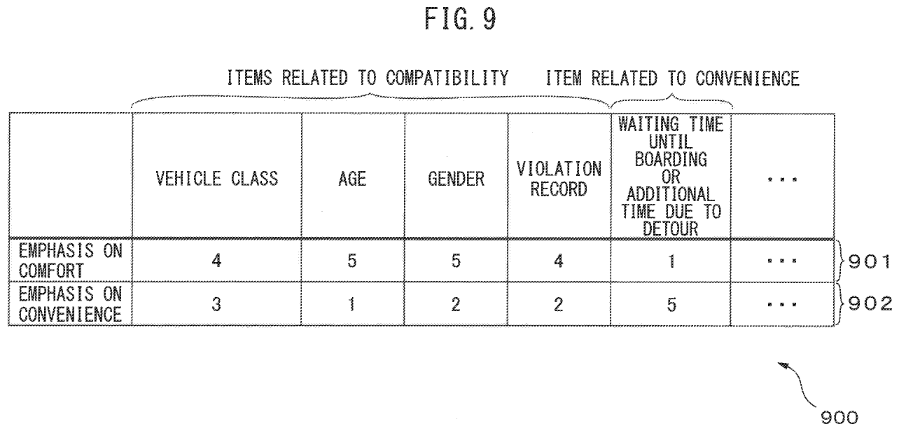

FIG. 9 is a diagram illustrating an example of a set of weighting factors for each of the case where emphasis is placed on comfort and the case where emphasis is place on convenience. In the table 900 illustrated in FIG. 9, a set of weighting factors 901, which is shown in the upper row, is a set of weighting factors for the case where emphasis is placed on comfort while a set of weighting factors 902, which is shown in the lower row, is a set of weighting factors for the case where emphasis is placed on convenience. In the set of weighting factors 901, comfort for each user is considered to improve as compatibility between the users is better; therefore, weighting factors for items related to compatibility (i.e., items related to comfort) such as a class of a vehicle owned, age, gender, or a violation record is set to a relatively greater value compared with weighting factors for items related to convenience such as an additional time due to sharing a ride or a waiting time until the meeting time. On the other hand, in the set of weighting factors 902, weighting factors for items related to convenience is set to a relatively greater value compared with weighting factors for items related to compatibility.

According to the variation, the ride-sharing assistance system can determine, according to a length of a section for which users share a ride or a period of time for which the users share a ride, a combination of users who will share the ride more appropriately.

According to still another variation, each terminal may include a current heart rate or a current blood pressure of a user received from a biological sensor attached to the user in the ride-sharing request signal. When the processor 24 of the server 2 calculates the evaluation score for each combination of users, the processor 24 may refer to the heart rate or the blood pressure included in the ride-sharing request signal. In this case, the processor 24 may set the evaluation score to be lower, for example, as the heart rate is higher or the blood pressure is higher. With this operation, a possibility of suggesting ride-sharing for a combination including a user whose physical condition may be disturbed during the ride-sharing will be reduced.

According to yet still another variation, the processor 24 of the server 2 may compare the highest value of the evaluation scores calculated for each combination of users with an evaluation score threshold set in advance, and determine not to suggest ride-sharing when the highest value is lower than the evaluation score threshold. With this operation, when compatibility between users is not good for any combination, the ride-sharing assistance system may prevent the users from sharing a ride on a single vehicle and prevent a situation in which the users feel stress due to the ride-sharing.

According to yet still another variation, each of terminals 3-1 to 3-n may include at least one of information about a vehicle owned by a user of the terminal and personal information of the user in a ride-sharing request signal. In this case, the memory 34 of each terminal may store the information about the vehicle owned by the user of the terminal and the personal information of the user in advance. When the processor 35 of each terminal generates the ride-sharing request signal, the processor 35 may read out, from the memory 34, the at least one of the information about the vehicle owned by the user of the terminal and the personal information of the user, and include the at least one in the ride-sharing request signal. Alternatively, the user of each terminal may operate the user interface 31 in accordance with an application program associated with the ride-sharing assistance process, and input at least one of the information about the vehicle and the personal information, and the processor 35 may include the at least one of the information about the vehicle and the personal information in the ride-sharing request signal, which has been input. With this operation, even if the information about the vehicle owned by the user and the personal information has not been registered in advance, the server 2 can calculate the evaluation score representing compatibility between users; therefore, the server 2 can determine whether to suggest ride-sharing and a combination of users who will share a ride.

According to yet still another variation, the amount of the price for ride-sharing may be defined according to a vehicle used for the ride-sharing. For example, the amount of the price for the ride-sharing may be defined in such a way that the amount of the price for the ride-sharing is higher as a purchase price of the vehicle used for the ride-sharing is higher, or as a class of the vehicle used for the ride-sharing is more upper. With this operation, it is expected that the user of the vehicle used for the ride-sharing is more satisfied with the amount of the price for the ride-sharing.

According to yet still another variation, a server for managing points of a user of each terminal (for distinction from the server 2, hereinafter referred to as a point management server) may be provided separately from the server 2. The point management server may be capable of communicating with the server 2 via the communication network 4. In this case, in processing of a portion associated with the ride-sharing assistance process illustrated in FIG. 5, processing for the step S210 may be omitted. Instead, the processor 24 of the server 2 may generate a signal for informing points to be added to the points of the user of the vehicle used for the ride-sharing and points to be subtracted from the points of the user who shared the ride, and send the signal to the point management server via the communication interface 21 and the communication network 4.

The computer program that causes a computer to execute the process to be executed by the processor 24 of the server 2 as described above may be recorded and distributed on a recording medium, for example, an optical recording medium or a magnetic recording medium.

As described above, those skilled in the art may make various modifications according to embodiments within the scope of the present invention.

What is claimed is:

1. A ride-sharing assistance apparatus, comprising:
a communication circuit capable of communicating with a plurality of terminals; and
a processor configured to:
calculate, upon receiving a ride-sharing request signal indicating a request for ride-sharing on a vehicle from at least two terminals of the plurality of terminals via the communication circuit, an evaluation score, for each combination of users of the at least two terminals, that represents compatibility between users included in the combination, based on information about a vehicle owned by each of the users of the at least two terminals,
determine a combination of users having the highest evaluation score as a combination of users who will share a ride on a vehicle, and
send a suggestion signal for suggesting ride-sharing on a vehicle to terminals of the users included in the combination of the at least two terminals via the communication circuit.

2. The ride-sharing assistance apparatus according to claim 1, wherein the information about the vehicle includes information indicating a class of the vehicle, and wherein the processor sets the evaluation score for a combination of users to be higher as the vehicles of the users belong to classes close to each other.

3. The ride-sharing assistance apparatus according to claim 1, wherein the processor sets the evaluation score to be lower as a heart rate or a blood pressure of a user who is included in the combination of users for which the evaluation score is calculated and who will share a ride on a vehicle is higher.

4. The ride-sharing assistance apparatus according to claim 1, wherein the processor calculates the evaluation score by performing a weighted addition of a first score, which is obtained, based on information about a vehicle owned by each user included in the combination of users for which the evaluation score is calculated, and which becomes higher as comfort for each user is better, and a second score, which becomes higher as convenience for each user in a case where the users included in the combination share a ride is greater.

5. The ride-sharing assistance apparatus according to claim 4, wherein, with respect to the combination of users for which the evaluation score is calculated, the processor is further configured to search, based on current locations and destinations of the users included in the combination, for a section for which the users will share a ride; when a length of a searched section or an estimated time required for the searched section is less than a predetermined threshold, the processor sets a higher weight for the second score than that for the first score; on the other hand, when the length of the searched section or the estimated time required is greater than the predetermined threshold, the processor sets a higher weight for the first score than that for the second score.

6. A ride-sharing assistance system including a plurality of terminals and a ride-sharing assistance apparatus capable of communicating with each of the plurality of terminals,
wherein the ride-sharing assistance apparatus calculates, upon receiving a ride-sharing request signal indicating a request for ride-sharing on a vehicle from at least two terminals of the plurality of terminals, an evaluation score, for each combination of users of the at least two terminals, that represents compatibility between users included in the combination, based on information about a vehicle owned by each of the users of the at least two terminals, determines a combination of users having the highest evaluation score as the combination of users who will share a ride on a vehicle, and sends a suggestion signal for suggesting ride-sharing on a vehicle to terminals of the users included in the combination of the at least two terminals.

7. A ride-sharing assistance method in a ride-sharing assistance system including a plurality of terminals and a ride-sharing assistance apparatus capable of communicating with each of the plurality of terminals, comprising:
sending, by at least two terminals of the plurality of terminals, a ride-sharing request signal indicating a request for ride-sharing on a vehicle to the ride-sharing assistance apparatus, and
calculating, by the ride-sharing assistance apparatus, an evaluation score, for each combination of users of the at least two terminals, that represents compatibility between users included in the combination, based on information about a vehicle owned by each of the users of the at least two terminals from which the ride-sharing request signal is received, determining, by the ride-sharing assistance apparatus, a combination of users having the highest evaluation score as a combination of users who will share a ride on a vehicle, and sending, by the ride-sharing assistance apparatus, a suggestion signal for suggesting ride-sharing on a vehicle to terminals of the users included in the combination of the at least two terminals.
